(19) United States
(12) Patent Application Publication
(10) Pub. No.: US 2005/0085552 A1
(43) Pub. Date: Apr. 21, 2005

(54) Method for providing long-lasting pain relief through intrathecal administration of civamide

(75) Inventor: Joel E. Bernstein, Deerfield, IL (US)

Correspondence Address: JONES DAY, 77 WEST WACKER, CHICAGO, IL, 60601-1692, US

(73) Assignee: Winston Laboratories, Inc., Vernon Hills, IL (US)

(21) Appl. No.: 10/686,880

(22) Filed: Oct. 16, 2003

(51) Int. Cl. A61K 31/16

(52) U.S. Cl. 514/625

(57) ABSTRACT

A method of treating or preventing painful disorders comprises the intrathecal administration of civamide or one of its salts in an amount of about 0.001-1 mg by weight in a pharmaceutically acceptable vehicle to provide surprisingly long-lasting pain relief.

### BACKGROUND OF THE INVENTION

Civamide (cis-8-methyl-N-vanillyl-6-nonenamide), also known as zucapsaicin, is a stereoisomer of the chemical capsaicin which has been utilized over the last three decades to study a variety of neurophysiological processes. Civamide was previously found to be useful in the treatment of painful, inflammatory or allergic disorders, and was effective in such disorders, yet with significantly less of the localized burning and stinging associated with capsaicin's use. Such use of civamide is disclosed in U.S. Pat. No. 5,063,060 issued Nov. 5, 1991, which is incorporated herein by reference in its entirety.

U.S. Pat. No. 5,063,060 chiefly distinguished between capsaicin and civamide in that compositions containing civamide were “comparable in efficacy to compositions containing capsaicin, but with significantly less local adverse effects normally associated with capsaicin.”

The applicant of this patent, however, has more recently discovered several properties of civamide heretofore unknown, and which make civamide ideally suited as an agent for intrathecal relief of pain. Firstly, it had been thought that civamide, like capsaicin, was neurotoxic and could cause degeneration of neurons. Such potential neurotoxicity would, or course, virtually contradict civamide's administration by the intrathecal route. In fact, the applicant has discovered that, surprisingly, civamide demonstrates no significant neurotoxicity. Secondly, the applicant has found that civamide administered intrathecally is surprisingly much more effective at blocking pain than intrathecally administered capsaicin. Thirdly, the applicant has discovered that extremely low doses of civamide (much lower than cited in U.S. patent No. 5,063,060) given intrathecally are surprisingly quite effective in blocking pain. While a single dosage of civamide cited in U.S. Pat. No. 5,063,060 ranged from 0.1 mg to 100 mg, the applicant herein has discovered that the single intrathecal effective dosage of civamide ranges from about 0.001 mg to a high of about 1 mg. Fourthly, while U.S. Pat. No. 5,063,060 and all subsequent publications on civamide indicate that civamide should be administered on a daily basis (usually several times per day), the current invention provides that a single dose of civamide administered intrathecally is sufficient to block pain in postoperative patients for the entire postoperative period, and that a dose of intrathecal civamide administered as seldom as about once every month or two is sufficient to relieve or ameliorate chronic neuropathic pain such as postherpetic or diabetic neuropathy.

It is an object of this invention to provide a method of treating pain that provides long-lasting effectiveness such that painful disorders as post-surgical pain and chronic neuropathic pain can be successfully alleviated with either a single intrathecal dose or infrequent intrathecal doses of civamide, respectively.

The invention, thusly, includes a method comprising the intrathecal administration of compositions of civamide (cis-8-methyl-N-vanillyl-6-nonenamide) incorporated into sterile solutions or suspensions at very low dosages (about 0.001 mg to about 1 mg) of civamide. Surprisingly, the method provides for significantly greater pain relief than comparable capsaicin formulations, no neurotoxicity, and long-term effectiveness with a single dose or infrequent (monthly or bimonthly) doses.

### DETAILED DESCRIPTION OF THE PREFERRED EMBODIMENTS

In accordance with the invention, formulations are provided for use with the inventive method that incorporate civamide into sterile solutions or suspensions suitable for intrathecal administration such as cerebrospinal injection. In each of the foregoing formulations, civamide may be present in a single dosage of from about 0.001 mg to about 1 mg. The civamide can be present as the compound civamide or as a pharmaceutically acceptable salt thereof, such as a hydrochloride salt or an acetate salt. The civamide composition can be in the form of a suspension with a pharmaceutically acceptable suspension agent, such as dimethylsulfoxide or cyclodextrin. The composition will include a pharmaceutically acceptable vehicle suitable for introduction into the intrathecal space, such as normal saline. In a preferred form, the composition will be packaged in sterile ampules or vials. Civamide is synthesized according to a proprietary process and supplied by Winston Laboratories, Vernon Hills, Ill.

The instant invention comprises the method of instilling or injecting sterile solutions or suspensions of civamide or one of its salts into the cerebrospinal fluid in a single dose or very infrequent doses (monthly or bimonthly) in order to treat a variety of painful disorders including post-surgical pain and chronic neuropathic disorders such as postherpetic neuralgia, diabetic neuropathy, reflex sympathetic dystrophy, and post-mastectomy pain. The civamide or its salt will be present in each dose in the amount of about 0.001 mg to about 1 mg.

The method of the instant invention will be more readily comprehended from the following examples.

### EXAMPLES

### Example 1

Civamide in amounts of 1 Φg, 5 Φg, 10 Φg, 50 Φg, and 100 Φg was dispersed in both 10 Φl and 20 Φl of each of the following: 100% dimethylsulfoxide (DMSO), normal saline (0.9% w/v sodium chloride) with 10% DMSO as suspending agent, normal saline with 0.5% DMSO and 10% cyclodextrin, 10% cyclodextrin and 10% DMSO as suspending agent, and normal saline with 10% cyclodextrin suspending agent, and normal saline with 25% DMSO. In each case, the saline was 0.9% USP. These compositions were physically and chemically stable, and used for injection into the cerebrospinal fluid of male Sprague-Dawley rats.

### Example 2

Civamide and capsaicin were each separately administered intrathecally, in dosages of 1 Φg, 5 Φg, 10 Φg, 50 Φg, or 100 Φg in either 10 Φl or 20 Φl of 0.9% USP saline with 25% DMSO suspending agent, 0.9% USP saline and 10% cyclodextrin, and 0.9% USP saline with 0.5% DMSO and 10% cyclodextrin, to male Sprague-Dawley rats into whom intrathecal catheters had been inserted. Seven days later, tail flick, hot plate (49° C., 52° C.) and paw pressure pain models were evaluated. In these pain models, intrathecally administered civamide was significantly more effective than saline, as well as more effective than intrathecally administered capsaicin.

### Example 3

Civamide 10 Φg/10 Φl saline, 25Φg/10 Φl saline, and 50 Φg/10 Φl saline and saline itself were administered intrathecally to male Sprague-Dawley rats. Each of the civamide compositions also included either 20% DMSO or 25% DMSO as a suspending agent. The saline used was 0.9% USP. Eighteen hours, 7 days, 14 days and 28 days after administration of a single intrathecal dose of either civamide or saline, models for various types of pain were evaluated. These included models for acute nociceptive processing (i.e. thermal escape), post tissue injury hyperpathic states (i.e. formalin and thermal injury evoked hyperalgesia) and nerve injury induced hyperpathia (i.e. tactile allodynia in the Chung model of neuropathy). The results of these studies demonstrated that within 18 hours after administration, intrathecal civamide produced effective pain amelioration, and the effects of a single dose lasted for at least 28 days after admistration.

While the foregoing is a description of the preferred embodiments of the invention, it will be readily apparent to those skilled in the art that various modifications may be made therein without departing from the true scope and spirit of the invention as set forth in the appended claims.

1. A method of treating or preventing painful disorders comprising the intrathecal administration of civamide (cis-8-methyl-N-vanillyl-6-nonenamide) or one of its salts in an amount of about 0.001 mg to 1 mg by weight in a composition comprising a pharmaceutically acceptable vehicle, such vehicle being suitable for introduction into the intrathecal space of humans or other mammals, in order to provide long-lasting prevention or relief of painful disorders.

2. The method of claim 1 wherein the dosages of intrathecal civamide are administered as a single one-time dose, or as single doses once about every 30 to 60 days.

3. The method of claim 1 wherein said method of treatment or prevention is utilized to prevent or treat post-surgical pain, chronic neuropathic pain or other types of chronic pain amenable to treatment or prevention with this method.
